(19) United States
(12) Patent Application Publication
(10) Pub. No.: US 2009/0304662 A1
(43) Pub. Date: Dec. 10, 2009

(54) Methods for Identifying and Targeting Tumor Stem Cells Based on Nuclear Morphology

(76) Inventors: William G. Thilly, Winchester, MA (US); Elena V. Gostjeva, Winchester, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/085,533

(22) PCT Filed: Dec. 8, 2006
(86) PCT No.: PCT/US06/47136

§ 371 Date: Jun. 11, 2009

Related U.S. Application Data
(60) Provisional application No. 60/748,951, filed on Dec. 9, 2005.

(51) Int. Cl. A61K 38/43; A61P 35/00

(52) U.S. Cl. 424/94.1

(57) ABSTRACT

Described herein are methods for inhibiting tumor growth comprising targeting a tumor stem cell in the patient with an agent or treatment that chemically modifies a tumor stem cell-specific molecule, thereby preventing proliferation of tumor stem cells.

### RELATED APPLICATION

This application claims the benefit of U.S. Provisional Application No. 60/748,951, filed on Dec. 9, 2005. The entire teachings of the above application are incorporated herein by reference.

### BACKGROUND OF THE INVENTION

Scientists have recognized the resemblance of tumor cells and pathological tissue constructs (such as teratocarcinomas) to the cells and tissues of early embryos. Normal but undifferentiated embryonic stem cells were able to give rise to organs by undefined processes that logically had to include rapid increase in cell number and differentiation to in organ anlage and subsequent organogenesis. Malignant tumors grow at rates similar to early fetuses and contain niches that are either histologically undifferentiated or organized with a histological appearance of normal tissue.

In addition to the complex antigenic glycosoaminoglycans at the cell surface, the “carcinoembryonic antigens”, molecules involved in cell adhesion and tissue restructuring, e.g., cadherins, catenins, metalloproteases, are expressed in both fetal tissues and tumors.

Tumors mimic the mitochondrial use of amino acids to reduce oxygen under the hypoxic conditions of early fetal tissues.

Oncogenesis like ontogenesis appears to proceed by lineal descent through an expanding set of stem cells. Only a small fraction of cells from a human tumor have the capacity to form new tumors as xenografts in immuno-suppressed rodents. Limiting dilution xenograft experiments have shown that one or more cells among the putative tumorigenic cells display stem cell-like properties in that they are capable of generating new tumors containing additional stem cells as well as regenerating the phenotypically mixed populations of cells present in the original tumor.

The concept of monoclonality of tumors was established in the early 1900's, and in the 20th it was determined that nearly all forms of late-onset cancer pass through an extended period of preneoplasia and that these preneoplastic colonies were themselves monoclonal and resulting from more than one rare cytogenetic mutation from the germinal DNA. By the beginning of 21st century, direct attempts to enrich tumor cell populations with “stem” cells for transplant/dilution experiments had demonstrated that not only were tissue stem cells the likely cells of origin of preneoplasia but tumors themselves contained “stem” cells. Modern restatement of the hypothesis that tumors are in fact reasonably well-organized heterogeneous fetal structures has been suggested. ‘Carcinoembryonic’ stem cells would be expected to increase in number and give rise to differentiated cell types populating the highly heterogeneous niches within the tumor mass. Recognition of cells as stem cells in developing organs and tumors, however, has not yet been accomplished.

Various antigenic markers employed throughout the stem cell field have been used to enrich for cells capable of regenerating tissues or tumors often to a high degree. No cells within these enriched populations, however, have demonstrated any microscopic morphological cellular property that marks them as stem cells. If it is true that tumors arise from a single stem cell, a means is required to identify them and to collect them as homogeneous population of stem cells sufficient for analysis of molecular and biochemical analytes. Only then can one focus the power of macromolecular array technologies (genomics, proteomics, glycomics etc.) and powerful forms of biochemical analysis such as magic angle nuclear magnetic resonance spectrometry.

### SUMMARY OF THE INVENTION

The invention is directed to methods of identifying tumor stem cells and selectively and specifically destroying tumor stem cells without or with minimal damage to normal or maintenance stem cells in their close environment. Disclosed herein is the unexpected finding that tumor stem cells contain nuclei where, during nuclear division, the genome is, for a significant and exploitable period of time, substantially single-stranded DNA (ssDNA). By using agents, e.g., chemical or enzymatic agents, that target and alter ssDNA (e.g., alkylating reagents, single-strand-specific nucleases, agents that target replication machinery, agents that target segregation and agents that target one or more stem cell-specific molecules), the nuclear material of tumor stem cells is targeted for destruction, as the modified ssDNA would be unable to undergo further replication back into double-stranded DNA (dsDNA). A tumor stem cell-specific molecule is a molecule present in tumor stem cells, preferably in the nucleus, and not in the cells of surrounding cells. Specific tumor-cell-specific molecules can be targeted, whereby targeting and disruption of the function or activity of the tumor stem cell-specific molecule prevents or inhibits tumor cell growth. For example, any agent that prevents replication of ssDNA, e.g., a molecule that hybridizes to DNA, but is incapable of being extended, e.g., a modified oligonucleotide or nucleic acid derivative, e.g., a nucleic acid lacking the γ-phosphate necessary for extension or a peptide nucleic acid. Methods are known in the art for delivering agents to cells or tumor tissue in a patient, where such agents would prevent replication of the ssDNA genome, and thereby prevent proliferation of the tumor stem cells.

In one embodiment, methods are directed to the selective prevention or inhibition of growth (e.g., nuclear or cellular division) of tumor stem cells without substantially preventing or inhibiting the growth of surrounding cells (e.g., maintenance stem cells). In particular, while the targeted stem cell is undergoing nuclear division, the method comprises contacting the cell with an agent capable of entering the nucleus of the cell and modifying or altering the ssDNA of the nucleus, resulting in the prevention or inhibition of further nuclear and cellular division of the targeted cell.

In another embodiment, methods are directed to a method for inhibiting tumor growth in a patient comprising targeting a tumor stem cell in the patient with an agent or treatment that alter or modifies a tumor stem cell-specific molecule (e.g., ssDNA), thereby preventing or inhibiting replication of ssDNA and ultimately preventing or inhibiting proliferation of tumor stem cells. In a particular embodiment, the agent targets a tumor stem cell-specific molecule that is synthesized within the cell and segregates into daughter bell-shaped nuclei. In one embodiment, the tumor stem cell-specific molecule is single-stranded DNA (ssDNA). In another embodiment, the agent is a chemical agent, radioagent, enzyme, or radiation treatment, whereby a tumor cell-specific molecule is targeted.

### BRIEF DESCRIPTION OF THE DRAWINGS

FIG. 1 is a summary of key images. A) Examples of nuclear morphotypes observed in interphase and early prophase (E.P.) cells in human fetal gut, normal colonic mucosa, adenomas and adeno-carcinomas. B) High resolution image (×1400) of bell-shaped nuclei of fetal gut. Condensed DNA appears to create an anulus that maintains an opening into the hollow bell structure. Scale bar, 5 μm.

FIGS. 2A and B are images of Embryonic gut, 5-7 weeks. FIG. 2A: Phase-contrast image (left frame) and stained nuclei image (middle) and the merged image (right) show the linear arrays of nuclei within ˜50 micron diameter tubular syncytium. FIG. 2B: High resolution image of the nuclei shows hollow bell-shaped structures The ‘head to toe’ orientation of the bells is preserved in all embryonic tubes observed but tubes snake backwards and forwards such that parallel tubes may have locally anti-parallel bell-shaped nuclei orientation. Scale bars, 50 μm at low and 5 μm at high magnification.

FIGS. 3A-D show images of nuclear fission of bell-shaped nuclei in fetal gut. FIGS. 3A and B: Symmetrical nuclear fission. Bell-shaped nuclei emerges from bell-shaped nuclei of similar shape. FIGS. 3C and D: Asymmetrical nuclear fission. A spherical nucleus, and a ‘cigar’-shaped nuclei emerging from a bell-shaped nucleus. Scale bar, 5 μm.

FIGS. 4A-C show images from normal adult colonic crypts. FIG. 4A: Crypts of about 2000 spheroid, spherical or discoid nuclei occasionally (< 1/100) contained a recognizable bell-shaped nucleus (arrow) located at the bottom of the crypt. FIG. 4B: Crypt base showing another bell-shaped nucleus. FIG. 4C: Morphotypes of interphase and mitotic nuclei of the walls and luminal surface in a well-spread crypt. The enlarged images show: (i) spherical and ovoid interphase nuclei, (ii, iii) early prophases of spherical- and oval-shaped nuclei, and (iv) an anatelophase nucleus. Scale bars, 100 μm for low and 5 μm for high magnification images.

FIGS. 5A-E show images from Adenomas. FIG. 5A: Characteristic large branching crypt of adenomas. FIG. 5B: An irregular crypt-like structure found throughout adenomas. Typically two, but sometimes 1, 4 or even 8, bell-shaped nuclei (insert) appear at the base of these large (>4000 cell) irregular crypt-like structures. FIG. 5C: A cluster of cells of similar nuclear morphotype containing one bell-shaped nucleus. These forms of clusters contain exactly 16, 32, 64, and 128 total cells. Left panel, Feulgen-Giemsa stain. Right panel, phase contrast autofluorescent image. FIG. 5D: Contexts in which bell-shaped nuclei appear in adenomas: (i) Cluster with 31 ovoid nuclei and one bell-shaped nucleus, (ii) Multiple bell-shaped nuclei in shoulder to shoulder arrangement, (iii) Bell-shaped nuclei arranged in a side-by-side pattern (arrow) (iii). Irregular mixture of ˜250 nuclei of with several bell-shaped nuclei suggestive of nascent crypt bases. FIG. 5E: Irregular crypt-like structure containing apparently clonal patches of cells of 5 different nuclear morphotypes with one bell-shaped nucleus (arrow) at the base. Scale bars, 100 μm (in ‘a,b’) and 5 μm (in ‘e’).

FIGS. 6A-E shows images from adenocarcinomas. FIG. 6A: Very large crypt-like structures (>8000 cells), with branches with frequent break points. The arrow indicates an example of an ˜250 cell crypt-like structure found primarily near the surface of the tumor. FIG. 6B: Interior tumor mass with multiple where multiple bell-shaped nuclei (˜3% of all nuclear morphotypes). FIG. 6C: Bell shaped nuclei in FIG. 6B oriented in head-to-toe syncytial and non-syncytial side-by-side configurations. FIG. 6D: Symmetrical nuclear fission in adenocarcinoma. FIG. 6E: Asymmetrical nuclear fission of a bell creating a cigar-shaped nucleus in adenocarcinoma. Similar structures have been observed in colonic metastases to the liver. Scale bar, 5 μm.

FIGS. 7A-D are illustrations of the stages in quantitative image cytometry in the study of in human tissues and cells. FIG. 7A: Fresh colon surgical discard ready for fixation. FIG. 7B: Microscopic slide preparation showing the result of spreading of 1 mm section through a polyp (positioning of a polyp, ‘top-to-bottom’ is outlined). FIG. 7C: Cell nuclei spreads (in magenta color) observable for the whole crypts. All of the crypt nuclei are preserved, as compared to 5μsections (BrdU staining and H&M staining), shown above. FIG. 7D: Motorized Axioscop microscope-AxioCam color CCD camera-KS 400 software image analysis workstation.

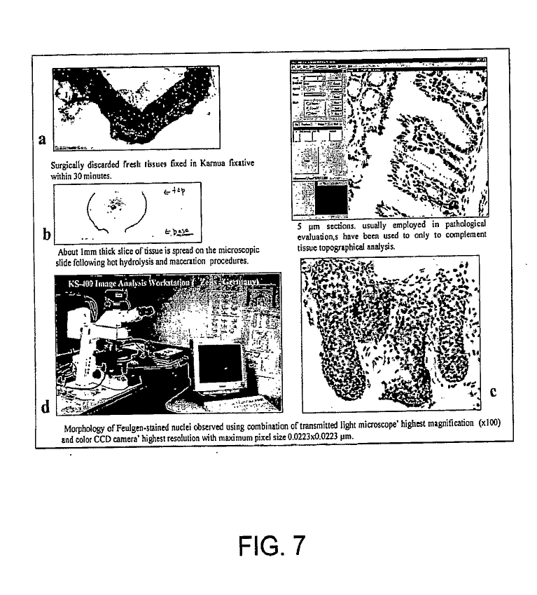

FIGS. 8A and B are illustrations of a ‘target of interest’ in application of FISH to explore non-dividing and dividing bell-shaped nuclei in tumors. FIG. 8A: Chromatin (stained darker because of higher DNA content per μm²) creates the unique structure resembling prophase chromosomes arranged as two parallel circles. These circles put into drawing illustrate the prediction of that specific chromosomes might be found at this specific site of bell-shaped nuclei. FIG. 8B: Chromatin distribution and specific chromosome positioning changes as imaginary transformation (‘bell-to-oval’ shaped nuclei here) taking place throughout asymmetrical division of the bell-shaped nuclei.

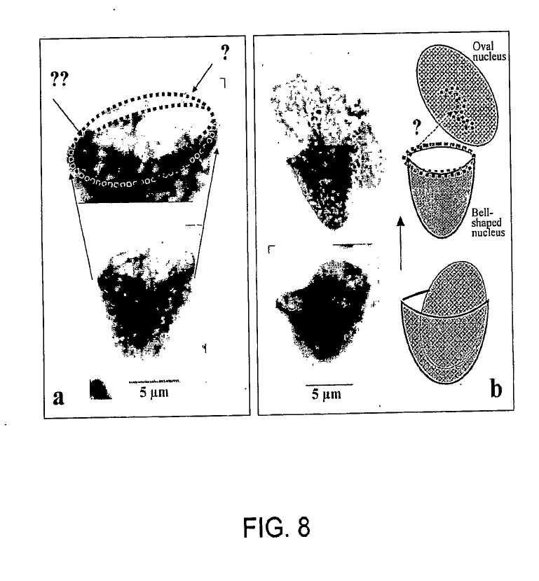

FIGS. 9A-D are images illustrating the results of fluorescent in situ hybridization of chromosome 11 in spherical nuclei of TK-6 human cells. FIG. 9A: two pairs of chromosomes in prophase chromosome spreads. FIG. 9B: spherical nuclei DAPI nuclear stain. FIG. 9C: same chromosome pair hybridized with FITC fluorescence probe. FIG. 9D: merged image of DAPI and FITC interphase chromosomes stain. Bar scale, 5 microns.

FIG. 10 shows images of symmetrical nuclear division of bell-shaped nuclei arranged in syncytia.

FIG. 11 shows images depicting the localization of DNA in bell-shaped nuclei undergoing nuclear division.

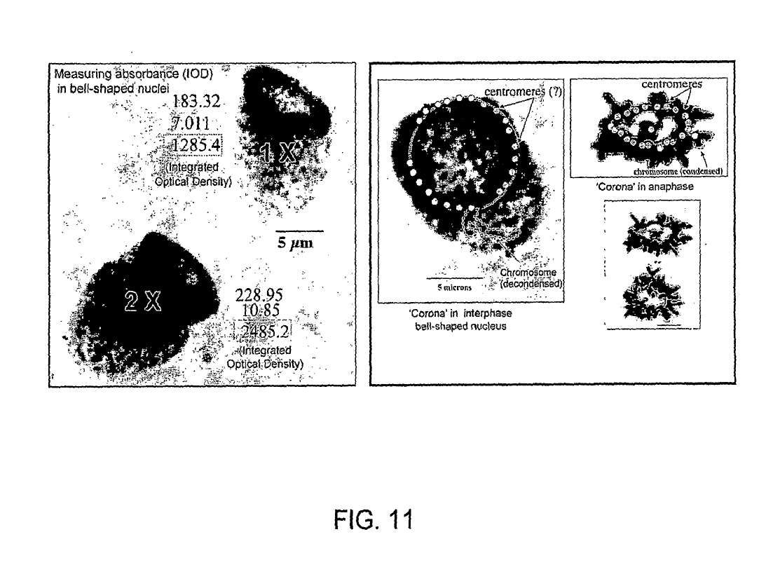

FIG. 12 shows arrangement and composition (ssDNA or dsDNA) of nuclear material during nuclear division of bell-shaped nuclei.

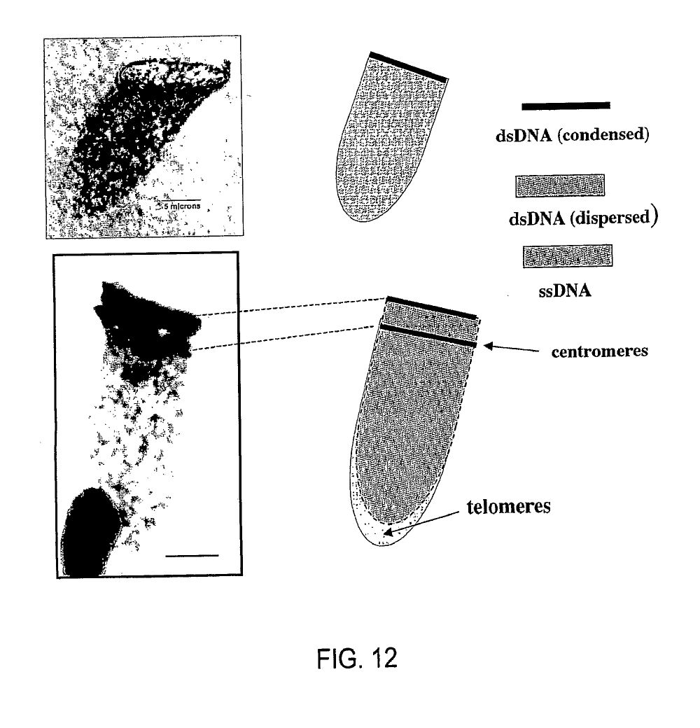

FIGS. 13A-D show images from human fetal preparations depicting a series of previously unrecognized nuclear forms. These forms give rise to the original bell-shaped nuclei. FIG. 13A shows a nucleus with a condensation of ˜10% of the total DNA content as a “belt” around the long axis of spherical or slightly oval nuclei. FIG. 13B shows a nucleus in which two condensed nuclear “belts” appear to have separated but are still part of a single nucleus. FIG. 13C shows a pair of nuclei that appear to have arisen by fission of the two-belted nucleus of FIG. 13B. FIG. 13D shows that each syncytium contains a set of bells with a single pair of bells at its linear midpoint with mouths facing as in FIG. 13C. These images show that a series of symmetrical divisions create nuclei pushing away from a central pair.

FIGS. 14A and B show nuclear morphotypes in colonic adenomas (FIG. 14A) and adenocarcinomas (FIG. 14B). Morphotypes of carcinogenesis show similar belts—one or two around the long axis of oval nuclei.

FIGS. 15A-C show FISH staining specific for human centromeres. FIG. 15 shows centromeres (bright) in spherical (FIG. 15A), “cigar”- (FIG. 15B) and bell- (FIG. 15C) shaped nuclei from tissues of human 12 weeks fetal colon.

### DETAILED DESCRIPTION OF THE INVENTION

The presented invention is related to the unexpected discovery that tumor stem cells, e.g., cells that divide leading to tumors, undergo asymmetric nuclear division. Bell-shaped nuclei, not found in adult tissues except for tumor tissues, undergo periods of time where the genome is represented as single-stranded DNA (ssDNA). This feature of asymmetrically dividing bell-shaped nuclei allows for the specific targeting of cells containing such nuclei, e.g., tumor stem cells, for identification and destruction.

Structures with Bell-Shaped Nuclei have Stem Cell-Like Qualities in Human Tumors.

Described herein are methods that build upon the unexpected discovery that bell-shaped nuclei divide both symmetrically and asymmetrically by non-mitotic fission processes in colonic and pancreatic human tumors (Gostjeva et al, 2005, Cancer Genetics and Cytogenetics, in press). These bell-shaped nuclei appear in great numbers both in 5-7 week embryonic hindgut where they are encased in tubular syncytia and comprise 30% of all nuclei and tumor tissues where they abound in “undifferentiated” niches. They possess several stem cell-like qualities, particularly the “shibboleth” of asymmetric division and a nuclear fission frequency consistent with growth rates of human colonic preneoplastic and neoplastic tissue (Herrero-Jimenez et al., 1998, 2000). These previously unrecognized nuclear forms are both the source of tumor generation and differentiation and thus targets for cancer therapeutic strategies.

Structures (e.g., cells, cell-like structures or syncytia) containing bell-shaped nuclei represent the tumor stem cells. Their amitotic mode of nuclear fission requires molecular machinery that would define molecular targets that are not expressed in embryonic (blastomeric) and adult maintenance stem cells that appear to divide by mitosis. For example, the observation that these bell-shaped nuclei undergo a stage where the genome is represented as ssDNA would allow for their targeting and destruction. Exploration of how bell-shaped nuclei are spatially organized, how chromatin is dispersed in the nuclei, whether or not specific chromosomes occupy specific territories throughout the interior of nuclear lamina will suggest more specific therapeutic targets research and provide additional understanding of the relationship between nuclear morphotype (shape) and gene expression.

Disclosed herein is the discovery of an array of distinct closed nuclear forms in fetal hindgut, colonic adenomas and adenocarcinomas that appear to arise ab initio from asymmetrical nuclear fission from bell-shaped nuclei but subsequently divide by mitosis and die by apoptosis. The shared set of nuclear forms in embryos and tumors that are absent in adult tissue support the 19th century hypothesis that tumors were embryonic growths in adult organs (Cohnheim, J., Virchows Arch., 65: p. 64, 1875; Sell, S., Crit. Rev. One. Hematol., 51:1-28, 2004).

The methods described herein allow one of skill in the art to carry out in vivo analysis of cytogenetic end-points of the nuclei of different morphologies, with special emphasis on bell-shaped nuclei in colon, pancreatic, kidney, ovarian and other tumors, based on state-of-the-art high-resolution microcopy and quantitative image analysis techniques.

Nuclear structures, DNA content and the spatial distribution of chromosomes in bell-shaped nuclei of cells and syncytia can be characterized by methods known to one of skill in the art, e.g., by quantitative image cytometry and confocal microscopy. For example, such techniques allow one of skill in the art to determine total DNA content, and to use specific reagents, such as, for example, acridine orange or strand-specific DNA hybridization probes, to distinguish ssDNA from double-stranded DNA (dsDNA). This information can be used to distinguish bell-shaped nuclei of differing morphology, tumor type (colonic vs. pancreatic) and niches within tumors. Such techniques can also be used to characterize the progress of DNA synthesis and detect the presence of proteins associated with, for example, DNA synthesis and segregation in bell-shaped nuclei during symmetrical and the several forms of asymmetrical nuclear fission.

Isolation of Cells and Syncytia with Bell-Shaped Nuclei as Homogeneous Samples.

The methods described herein and elsewhere (PCT/US2005/021504, filed Jun. 17, 2005; and U.S. application Ser. No. 11/156,251, filed Jun. 17, 2005; the contents of which are incorporated herein by reference in their entirety) of tumor tissue preparation can be adapted to the requirements of “catapult” pressure activated laser micro-dissection to create samples of cells homogeneous for nuclear morphology that may be applied to analyses of metabolites and macromolecules. Such a method allows one of skill in the art to identify logical targets for slowing the division, or killing of structures containing bell-shaped nuclei in human tumors.

The methods of the present invention are based in part on means to recognize nuclear morphology in unfixed tumor preparations so that homogeneous preparations of live cells and syncytia with bell-shaped nuclei can be studied ex vivo. Live bell-shaped nuclei can be studied to better understand their peculiar DNA synthetic and segregation mechanisms and suggest means to interfere with these processes in cancer therapies.

### The ‘Stem Cell Target’ in Cancer Therapeutics.

The primary targets of existing methods of cancer therapeutics are cells transiting the cell cycle (Gomez-Vidal, J. et al., A., Curr. Top. Med. Chem., 4: 175-202, 2004; Fischer, P. and Gianella-Borradori, A., Expert Opin. Investig. Drugs, 14:457-477, 2005). No distinction is made between cells in transit between adult maintenance stem cells that divide to provide transition cells to replace the loss of terminal cells lost by programmed cell death and tumor stem cells. Therapy aims at the narrow window of regimens that kill all tumor stem cells without killing the patient. But adult maintenance stem cells would logically be expected to have the property of zero net cell growth while tumor stem cells, like fetal stem cells, are by definition involved in rapid net cell growth. Adult maintenance stem cell divisions would seem per force to be asymmetrical in nature giving rise to a new maintenance stem cell and a first differentiated transition cell. Tumor stem cells would require successive symmetrical nuclear divisions to support net tumor growth. It is in the discovery of bell-shaped nuclei undergoing symmetrical ‘cup-from-cup’ nuclear division in tumors that a specific target for cytostatic or cytocidal therapies has been found.

Independent of these cytocidal strategies of chemotherapy has been the hypothesis that tumors could by asphyxiated by preventing angiogenesis, e.g., Folkman and Ingber (Sem. Cancer Biol., 3:88-96, 1992). Others have suggested approaches to cancer therapy by blocking cellular differentiation. But creating hypoxia may recreate the conditions of early embryogenesis so far as stem cells are concerned and may explain the palliative but not curative effects of anti-angiogenic tactics (Warburg, O., Biochem. Zeitschrift, 152:479, 1924). Blocking differentiation in tumors may block differentiation in normal tissues with undesirable consequences. Understanding that current cancer therapies are only minimally effective because the stem cells can repopulate tumors in a short period of time has become a powerful stimulus to the search for molecular and biochemical characteristics peculiar to tumor stem cells as opposed to adult maintenance stem cells (Otto, W., J. Pathol., 197:527-535, 2002; Sperr, W. et al., Eur. J. Clin. Invest., 34 (Suppl 2):31-40, 2004; Venezia, T. et al., PLoS Biol., 2: e301, 2004). Such molecular and/or biochemical characteristics of tumor stem cells, could serve as targets in cancer therapeutics. The discovery of bell-shaped nuclei that undergo both symmetrical- and asymmetrical divisions in colonic tumors but not in adult colonic epithelium allows for one of skill in the art to differentiate tumor stem cells from adult maintenance stem cells (Gostjeva, E. et al., Cancer Genet. Cytogenet., 164:16-24, 2006).

### Cytogenetics of Stem Cells in Embryos Stem Cell Lines and Tumors.

Tumor stem cell properties include symmetrical divisions to achieve net stem cell growth and asymmetrical divisions to achieve self renewal and differentiation. However, the mechanisms of cell cycle progression, including DNA synthesis and segregation at nuclear fission, remain essentially unexplored in stem cells of embryos and tumors. This dearth of effort is understandable insofar as there have been no direct cytological markers to identify stem cells in human or tissues. Asymmetrical divisions in adult stem cells of murine colons and cell strains have been explored with the exceedingly important demonstration of selective pangenomic segregation of parental DNA strands in putative stem cells (Potten, C. et al., J. Cell Sci., 115:2381-2388, 2002; Merok, J. et al., Cancer Res., 62:6791-6795, 2002). This recognition of a stem cell-specific mode of genetic transmission of chromosomes in a non-random manner precedes the present recognition of what appears to be a stem cell-specific nuclear morphology and mode of divisions by several decades (Cairns, J., Nature, 255:197-200, 1975).

### EXAMPLES

### Example 1

### Establishing a Source of Tissues and Tumors

Adult tissue and tumor specimens were obtained as surgical discards by and from collaborators at the Massachusetts General Hospital, Department of Pathology (Gostjeva, E. et al., Cancer Genet. Cytogenet., 164:16-24, 2006). Use of anonymous discarded tumor and tissue sections has been approved by the MIT Committee on Use of Humans as Experimental subjects through the laboratory of Prof. W. G. Thilly.

### Development of a Method for Tissue Excision, Fixation, Spreading and DNA Staining.

The following protocol permits visualization of nuclei of tissue and tumor specimens of desirable clarity for structural and quantitative observations of chromosomes and nuclei. Key elements are use of fresh tumor samples fixed within 30 minutes of surgery and avoidance of standard procedure of thin sectioning. The bell-shaped nuclei are apparently early victims of autolysis in tissue and tumor samples and are no longer discernable some 45 minutes after resection. Standard 5 micron sections simply slice through the several nuclear forms discovered nearly all of which have minimum diameters greater than 5 microns. The specific technique devised is as evidence of significant progress:

Within 30 minutes after resection sheets (˜1 cm²) such as stripped colonic mucosa or ˜1 mm thick sections of adenomas, adenocarcinomas or metastases are placed in at least three volumes of freshly prepared 4° C. Carnoy's fixative (3:1, methanol:glacial acetic acid). Fresh fixative is replaced three times (every 45 minutes) and then replaced by 4° C. 70% methanol for sample storage at −20° C. Fixed sections are rinsed in distilled water and placed in 2 mL of 1N HCl at 60° C. for 8 minutes for partial hydrolysis of macromolecules and DNA depurination. Hydrolysis is terminated by rinsing in cold distilled water. The rinsed sample is steeped in 45% acetic acid (room temperature) for 15 to 30 minutes for “tissue maceration” that allows spreading and observation of plant and animal tissue sections with gentle pressure on microscope cover slips. Each macerated section is bisected into ˜0.5×1 mm pieces and transferred with 5 μL of acetic acid to a microscope slide under a cover slip. For tissue spreading 5 layers of filter paper are placed on the cover slip. A tweezers handle is moved steadily in one direction along the filter paper with slight and even pressure. In well-spread colonic tissue there are no damaged nuclei while crypts are pressed into what is essentially a monolayer. Cover slips are removed after freezing on dry ice and slides are dried for one hour. Slides are placed in Coplin jars filled with Schiff's reagent to stain partially depurinated DNA (Feulgen staining) at room temperature for one hour, rinsed in the same Coplin jar two times with 2×SSC (trisodium citrate 8.8 g/L, sodium chloride 17.5 g/L), once for 30 seconds and once quickly. Slides are then rinsed with distilled water and are suitable for image analyses of nuclei (Gostjeva, E., Cytol. Genet., 32:13-16, 1998). To achieve superior resolution, slides are further stained with Giemsa reagent. Immediately after rinsing in 2×SSC slides are placed in 1% Giemsa solution (Giemsa, Art. 9204, Merck) for 5 minutes then rinsed quickly, first in Sörenssen buffer (disodium hydrogen phosphate dihydrate 11.87 g/L, potassium dihydrogen phosphate 9.07 g/L), and then distilled water. The slides are dried at room temperature for one hour and placed in a Coplin jar filled with xylene for at least 3 hours to remove fat. Cover slips are glued to the slides with DePex mounting media and dried for 3 hours prior to high resolution scanning.

Alternatively, maceration can be achieved by exposure to proteolytic enzymes such as, for example, collagenase II, to achieve isolation of live cells with a defined nuclear morphotype.

### Microscope and Image Processing System.

The software for quantitative image analysis that is used herein utilizes an approach to background suppression adapted from earlier satellite surveillance systems. This technology was acquired by the Kontron corporation in Germany that has since been itself acquired by Zeiss, Inc. All images have been obtained using a customized KS-400 Image Analysis System™ Version 3.0, (Zeiss, Germany) consisting of a motorized light microscope, Axioscope™, color CCD camera, AxioCam™ (Zeiss, Germany) linked to a personal computer. Images are transmitted from the microscope at 1.4/100 magnification of the planar apochromatic objective using visible light and a 560 nm (green) filter when Feulgen stain alone was employed. No filter is used when Feulgen-Giemsa staining is employed. The frame grabber and optimal light exposure are adjusted prior to each scanning session. Nuclear images are recorded at a pixel size 0.0223×0.0223 microns.

### Embryonic Gut.

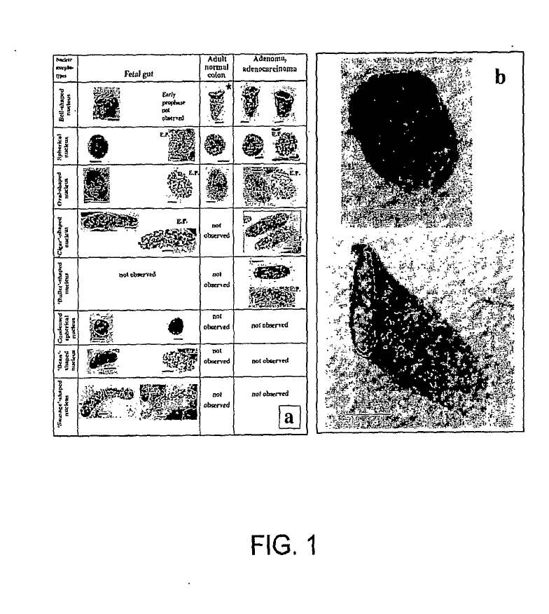

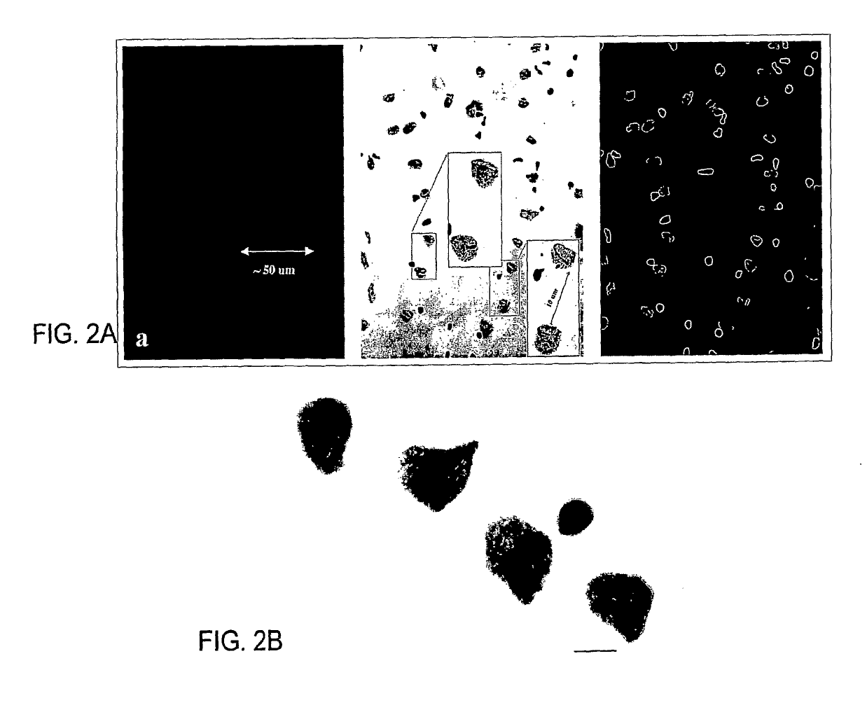

Seven distinct nuclear morphotypes (large spheroid, condensed spheroid, ovoid, bean-, cigar-, sausage- and bell-shaped) were found throughout the fetal gut samples (FIG. 1A). The bell-shaped nuclei that seemed to be held open by condensed chromatin resembling condensed chromosomes (FIG. 1B). Bell-shaped nuclei were organized in a linear ‘head-to-toe’ orientation within ˜20-50 micron tubes, or syncytia (FIG. 2). The ‘head-to-toe’ pattern of the bell-shaped nuclei was preserved in all embryonic tubes observed but tubes snaked backwards and forwards such that parallel tubes had locally anti-parallel orientation of bell-shaped nuclei.

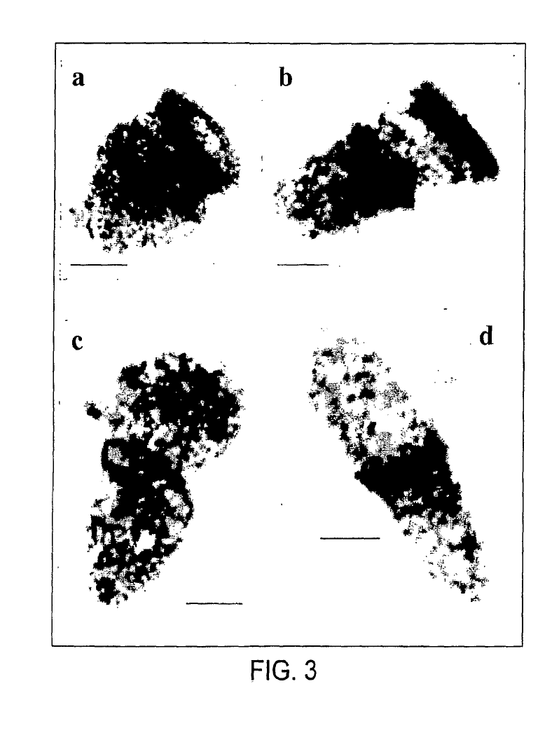

Bell-shaped nuclei were observed to undergo symmetrical and asymmetrical amitoses but only within the syncytia (FIG. 3). Symmetrical amitoses of bell-shaped nuclei resembled a simple separation of two stacked paper cups. At the highest resolution, condensed chromatin resembling paired chromosomes appeared to form an annulus that maintained the bell “mouth” in an open condition. Outside of the tubular syncytia mitoses were frequently observed for each of the several “closed” nuclear morphotypes and small colonies were evident consisting of cells of identical nuclear morphotype. Specific “closed” nuclear morphology was preserved in early prophase as shown in FIG. 1.

### Normal Colonic Epithelium.

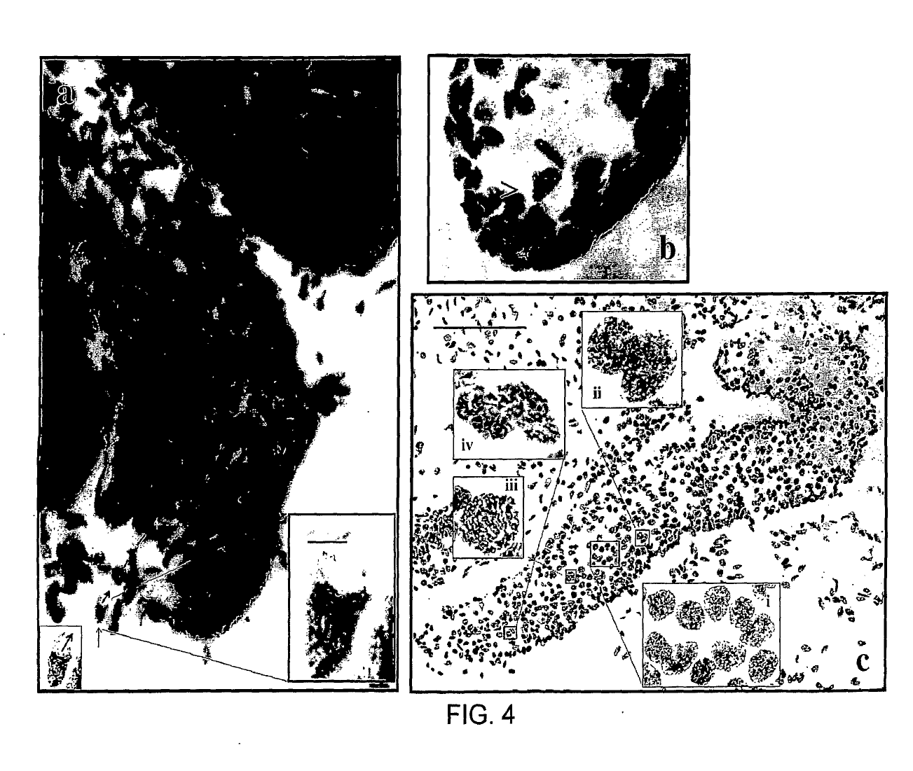

Nearly all nuclei in crypts could be observed from the crypt base to the luminal surface (FIG. 4A). Many crypts spread in such a way that individual nuclear shapes could be discerned. Cells with ovoid or spheroid nuclei line the crypt from just above the base to the epithelial extension into the lumen. (FIG. 4C). In the first ˜2⁵ cells of the crypt base, a potentially distinct, ninth nuclear morphotype predominated that may be characterized as discoid, ˜2-3 microns thick and ˜10 microns diameter (FIG. 4B). In less than 1% of all crypt bases in which the cells were well separated a solitary bell-shaped nucleus was discerned among the apparently discoid nuclei (FIGS. 4A and 4B). A similar low frequency of bell-shaped nuclei has been observed in preparations of adult liver. In an adult colon without any pathological indication of neoplasia or preneoplasia no other nuclear morphological variant was observed in a cell-by-cell scan of more than a thousand well spread crypts.

### Adenomas.

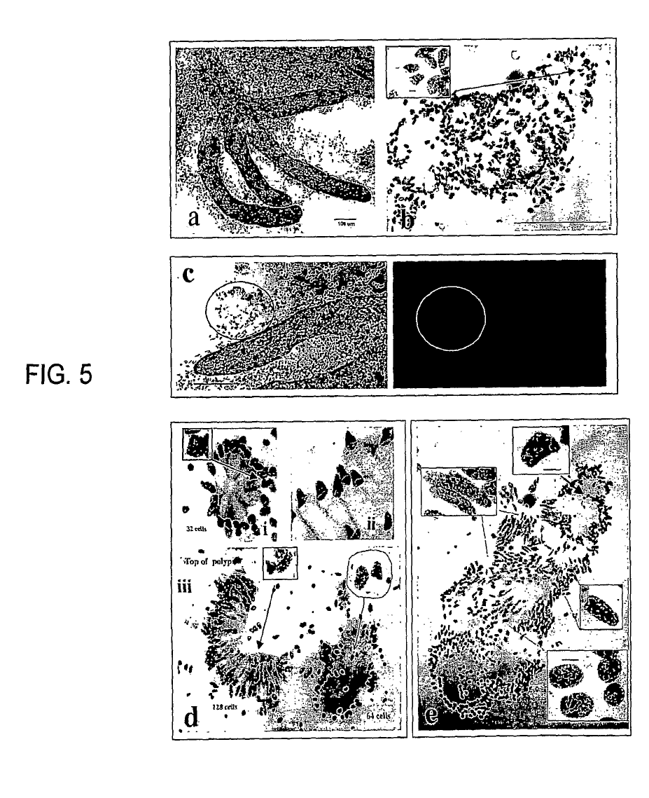

Adenomas contained many crypts, indistinguishable from normal colonic crypts each with ˜2000 cells. These were frequently found in branching forms as shown in FIG. 5A. The same spheroid and ovoid nuclei in the crypt walls as in the normal colonic crypts but more frequently than in the normal colon there were one or two bell-shaped nuclei in the crypt base. Irregular lobular structures were also observed containing up to ˜8000, cells the cells of which were more easily spread by tissue maceration. In nearly all of the irregular structures there were two or more bell-shaped nuclei oriented with the bell openings in the direction of the body of the structure (FIG. 5B). In addition many diverse cells and groups were interspersed among the crypts and irregular structures (FIG. 5C). Some regular structures appeared to be growing toward full-sized normal crypts containing ˜250, ˜500 or ˜1000 cells. Many cell groups were seen as “rings” of exactly 8, 16, 32, 64 and 128 cells each with one bell-shaped nucleus (FIG. 5D).

Higher magnification examination revealed that while most of the cells of the walls of the crypt-like structures had spherical or ovoid nuclei as in the normal adult colonic crypt. Colonies of cells with either ovoid, cigar- or bullet-shaped nuclei appeared in the irregular lobular structures suggesting a fusion of several different colonies. Colonies with ovoid and cigar-shaped nuclei had been observed in embryonic hindgut but the bullet-shaped nuclear morphotype was seen only in adenomas and adenocarcinomas (FIG. 5E). The bullet-shaped nuclear morphotype also arose from bell-shaped nuclei by asymmetrical amitoses with the irregular end emerging first. Small colonies of cells with bullet-shaped nuclei were seen and these colonies contained cells undergoing ordinary mitoses save for the interesting fact that the peculiar nuclear morphologies were retained to some extent from prophase through anatelophase.

While rare in the normal adult colon, the bell-shaped nuclei appeared frequently and in a number of adenoma contexts. Some were found as one to ten or more “bells” in the spaces among the crypt-like structures (FIG. 5D). Others were found as single “bells” in multicellular ring structures in which one bell nucleus was always seen in the ring with (2ⁿ−1) cells of spheroid or other morphotype (FIGS. 5C and 5D).

Bell-shaped nuclei appeared as single bells, more often as a pair of bells or occasionally 4 or 8 bells within the crypt-like structures basal cup. In the much larger irregular lobular structures, bell-shaped nuclei were anatomically integrated into the walls of the aberrant structures mixed with cells of other nuclear morphologies. It appeared as if these larger irregular crypt-like structures were mosaics of multiple different kinds of clusters each with it's own nuclear morphotype. Large adenomas (˜1 cm) were estimated to contain about 1000 bell-shaped nuclei. Hundreds bell-shaped nuclei have been observed in each of multiple adenomas but not a single bell-shaped nucleus in any adenoma has been observed in the symmetrical form of nuclear fission frequently found in embryonic sections; several examples of asymmetrical nuclear fission have been observed however, in adenomas.

### Adenocarcinomas.

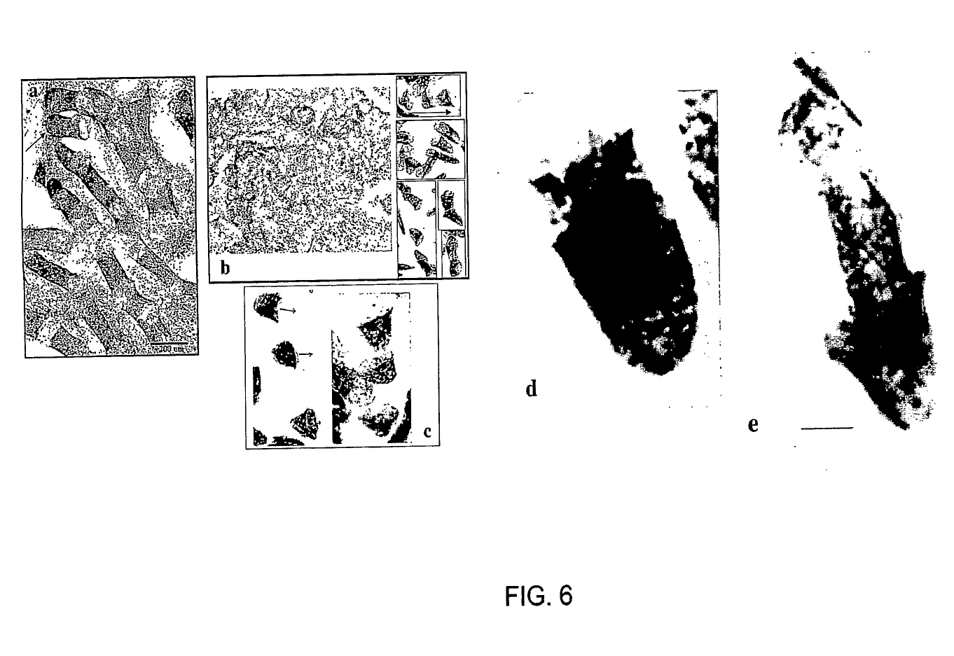

Adenocarcinomas like adenomas contained the admixture of crypts, larger irregular structures and inter-cryptal clusters of 16, 32, 64 and 128 cells. Bell-shaped nuclei were still found as singlets, pairs or larger numbers in the basal cup of crypts and embedded in complex whorls in the walls of the larger irregular lobular structures (FIG. 6). The set of nuclear morphotypes in the adenocarcinomas appear to be identical with the set seen in adenomas including the bullet-shaped morphotype.

A discernible difference between adenomas and adenocarcinomas was that the crypt-like structures were randomly oriented with regard to the tumor surface. Also crypts and irregular structures were not found frequently in the tumor interior, which may be better characterized as an eclectic but not chaotic collection of smaller, locally organized structures.

The most noticeable difference by which the adenocarcinomas differed from adenomas was the frequent appearance of apparently organized groupings of more than hundreds of bell-shaped nuclei many of which were frequently (˜1%) involved in symmetrical nuclear fissions. These symmetrical fissions were later identified as comprising condensed nuclear material. A bell-shaped nucleus would have an amount of DNA equal to that of a normal haploid cell. As the bell-shaped nuclei begin to undergo the “cup-from-cup” symmetrical division, the DNA content increases to 1.05 the amount of DNA contained in a haploid genome (approximately the increase one expects if the centromeres are replicated). The DNA content remains at this level until much later in the “cup-from-cup” process at which point the two nuclei contain 2 times the amount of DNA material. It is during the stage when perhaps only the centromeres have replicated, and the strands of the genome are separated that the genome is organized primarily into ssDNA. Not until replication much later in the process does the genome become dsDNA again.

At low magnification these structures appeared in the spaces among crypt-like structures and looked like a spider web or leaf vein skeleton. At higher magnification the thin veins were found to be partially ordered strands of cells with bell-shaped nuclei having the curious characteristic of having their mouths oriented in the same direction, 90° from the vein axis (FIG. 6C). Bell-shaped nuclei were also found in locally delimited syncytia in the ‘head-to-toe’ orientation (FIG. 6C) observed in the embryonic gut but not in the adenomas. Millions of bell-shaped nuclei are estimated to be in an adenocarcinomatous mass with frequent symmetrical and asymmetrical amitoses (FIGS. 6D and 6E). Metastases of colorectal tumors to the liver recreated the pattern of nuclear morphotypes, crypts and irregular structures seemingly indistinguishable from those observed for adenocarcinomas.

### Confocal Microscopy on 3D Preserved Single Bell-Shaped Nuclei and Pairs of Symmetrically Dividing Bell-Shaped Nuclei.

To perform confocal microscopy on 3D preserved single bell-shaped nuclei and pairs of symmetrically dividing bell-shaped nuclei, DeltaVision® RT Restoration Imaging System at Imaging Center, Whitehead Institute is used. The system provides real-time 2D deconvolution and 3D Z projections for restoration of nuclei images.

Counterstaining of nuclear cytoplasm (FITC-phalloidin) and nuclear (DAPI) have been applied to explore the interior structure of the bell-shaped nuclei. The cells are spread on the slide following same procedure as for Feulgen staining: by ‘hydrolysis’ maceration. The difference is that fixations in two different fixatives is applied to compare the results: Carnoy's fixative (4° C.) and 3.7% formaldehyde for 15 min and blocking solution for 2 hours in 2% BSA (2 g), 0.2% nonfat milk (0.2 g), 0.4% triton X-100 (400 μL) in 100 mL of PBS (room temperature), the latter as recommended for fixations of live tissue cells. Microscopic slides with tissue spreads on it, after twice washing in PBS, are transferred to humidity chamber, 100 mL droplets of primary antibodies diluted appropriately in blocking solution are dropped to cover the entire area of the spread and coverslips are sealed on the top by rubber cement, placed into container wrapped in foil and placed in the humidity chamber in the cold room overnight. Unsealed slides then washed three times in PBS. The slides are taken out and 100 μL droplets of secondary antibodies and/or cell stains (e.g., FITC-phalloidin, DAPI) diluted appropriately in blocking solution are placed again to cover the area containing the cells spread and transferred to humidity chamber placed in container. The container/humidity chamber is sealed, wrapped in foil and placed at room temperature for 2 hours. Slides are washed five times in PBS and prepared in a way that each have 2-5 μL droplets of mounting media (anti-fades SlowFade, VectaSheild or ProLong). Coverslips are mounted making sure an excess PBS is removed (dabbing the corner of the coverslip on a paper towel). The number of bubbles formed during mounting are limited by introducing the edge of the coverslip into the mounting media prior to lowering it completely. Coverslip are sealed on the slide using nail polish and the slides stored in dark at 4° C. (or −20° C. for longer periods). The slides are visualized using DeltaVision® RT Restoration Imaging System.

The protocol of Feulgen-Schiff procedure, which has been demonstrated to be accurate for the cytochemical localization of DNA and stoichiometry, was used to measure nuclei DNA contents. The DNA content was measured in single nuclei by measuring absorbance of molecules of a Feulgen-DNA (dye-ligand) complex (Kjellstrand, P., J. Microscopy, 119:391-396, 1980; Andersson, G. and Kjellstrand, P., Histochemie, 27:165-200, 1971). Non-dividing (interphase) and dividing bell-shaped nuclei were measured by measuring optical density integrated over the entire area (IOD) of each individual nucleus using software adapted from KS 400 image analysis system (Zeiss Inc. Germany).

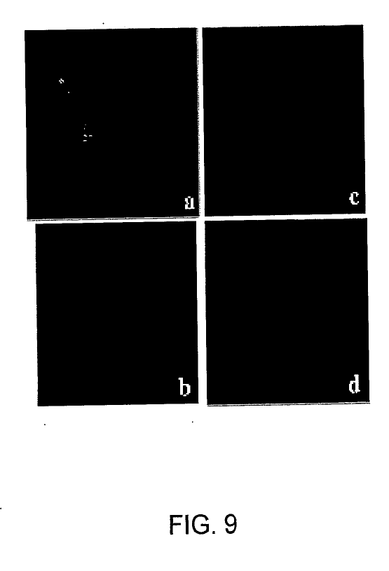

This particular image analysis workstation (See FIG. 9D) consists of a microscope Axioscop 2 MOT (Zeiss) coupled with AxioCam color CCD camera (Zeiss) connected to a computer, assembled by Carl Zeiss Inc. engineers, is capable of high-resolution image microscopy of nuclear and cell structures that is about 1000 bp of DNA per pixel in early prophase chromosomes measurements. Therefore, accurate measurements of condensed chromatin domains of ˜1 Mb pairs in interphase nuclei are possible. Images of the were scanned under constant parameters of magnification, light exposure and thresholding (contouring) of the nuclei using 560 nm green filter. This way of DNA content measurement was chosen as promising the most accurate results (Biesterfeld. S. et al., Anal. Quant. Cytol. Histol., 23:123-128, 2001; Hardie, D. et al., J. Histochem. Cytochem., 50:735-749, 2002; Gregory and Hebert, 2002; Gregory, 2005).

### Fluorescent In Situ Hybridization to Define the Spatial Distribution of all 24 Human Chromosomes in Bell-Shaped Nuclei in Interphase and During Nuclear Fission.

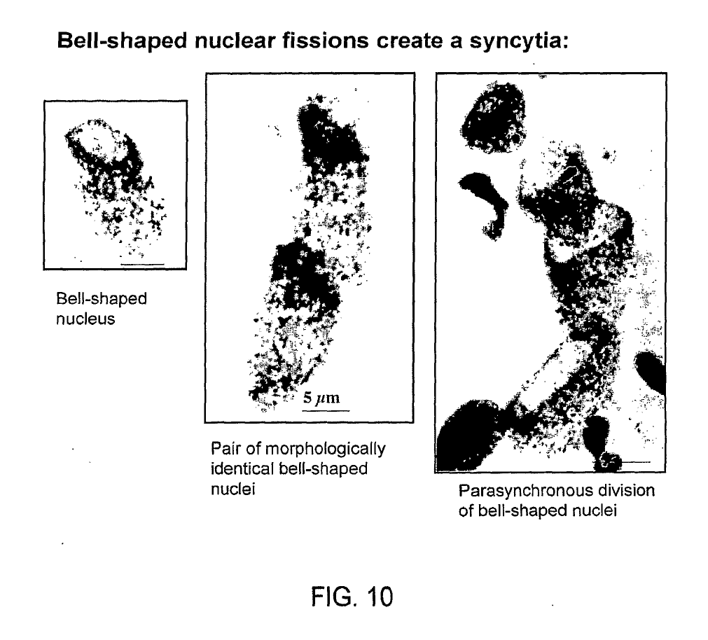

FISH was used to determine the whole chromosomes are involved in condensation that appears as a ‘ring’ on the top of the bell-shaped nuclei. Basically, labeling of chromosomes in the ‘ring’ is foreseen as a means to analyze their transformation when bell-shaped nuclei gives rise to a nucleus of different morphology (as shown in FIG. 10B) as well as developing of a fluorescence marker to recognize these nuclei by other means rather then nuclear morphology.

Tumor cells of not more then 1-5×10⁷ cells per slide are spread on the slide. The slides fixed in two different ways of cells spreading: one used in protocol for Feulgen DNA image cytometry and another proposed by Gibson for isolation of epithelial cells from colonoscopic biopsy specimens (Gibson, P. et al., Gastroenterology, 96:283-291, 1989). The latter is basically taking a tumor tissue within 30 min of surgery and immediately placing it in 50 mL of cold Hank's balanced salt solution, then washed. The specimens are then minced with a scalpel blade and digested for 1.5 h in 4 mL of collagenase-Dispase medium (culture medium containing 1.2 U/ml Dispase I (Boehringer Mannheim Biochemicals, Indianapolis, Ind.) and 50 U/ml collagenase type IV (Worthington, Biochemical Corp., Freehold, N.J.). The pellet is spread on the surface of microscopic slide by gentle sliding pressure on the coverslip. The spreading by ‘hydrolysis’ maceration serves as positive control to check if any distortion of bell-shaped nuclear morphology has occurred after applying collagenase-Dispase treatment for cells spreading. Prepared slides are dried out and put at 37° C. overnight. Slides then dehydrated sequentially in ice cold 70%, 80%, at room temperature 100% ethanol for 2 minutes each and dried completely, undergo denaturation in 70% formamide/2×SSC at 72° C. for 2 minutes and immediately dehydrated again with the same sequence and dried completely. Hybridization mixtures prepared that contains 7 μL hybridization buffer, 2 μL sterile water, and 1 μL probe. Mixtures are denatured at 72° C. for 8 to 12 minutes and immediately added to slides which then coverslipped, sealed with rubber cement, and put at 37° C. in a dark, humidified box overnight.

Slides are then dehydrated in cold 70% ethanol, cold 80% ethanol, and room temperature 100% ethanol for 2 minutes each; denatured in 70% formamide, 2×SSC at 72° C. for 50-60 seconds, depending on the extent of acetic acid denaturation. Slides are dehydrated again in cold 70% ethanol, cold 80% ethanol, and room temperature 100% ethanol for 2 minutes each. The hybridization mix includes 7 μL hybridization buffer, 1.5 μL sterile H₂O, and 1.5 μL. Whole Chromosome Paint probes (Vysis) with either Spectrum Orange or Spectrum Green fluorescent dye is applied. Hybridization mix is denatured for 5-10 minutes at 72° C. and slides subsequently dried completely. Hybridization mix is applied to the slides, coverslipped and sealed with rubber cement. Slides are then incubated overnight at 37° C. in a humidified box. On the following day, slides are washed in 50% formamide, 2×SSC at 42° C. twice for 8 minutes each. Slides are then washed with 2×SSC at 37° C. for 8 minutes and then washed three times in 1×PBD (0.05% Tween, 4×SSC) at room temperature for 1 minute each. Then 10 μL DAPI II Antifade, 125 ng/mL (Vysis) and coverslips is added. The excess DAPI II Antifade is blotted away and the slides sealed with rubber cement. Slides are kept in the dark at −20° C. prior to image scanning procedure.

Use of Quantitative DNA Cytometry to Track DNA Synthesis Before, During and after Nuclear Fission of Bell-Shaped Nuclei.

The techniques described herein permit detection of differences as low as 2% between any two nuclei or the anatelophases of sister nuclei during mitosis in human cell cultures. These techniques were used to determine when DNA is synthesized by cells or syncytia containing bell-shaped nuclei. This involved scanning nuclei that appear to be in the process of nuclear fission. It is noted that in general fetal bell-shaped nuclei containing the expected amount of DNA of a diploid human cell by comparison to human lymphocyte DNA content on the same stained slide. In addition, it is noted that the amount of DNA in bell-shaped nuclei of human preneoplastic lesions and tumors betrays a wide variation around a mean that is on average greater than the diploid DNA amount. Measurements have revealed another totally unexpected finding: DNA synthesis is concordant with rather than preceding the process of nuclear fission for both symmetrical and asymmetrical nuclear fissions involving bell-shaped nuclei. Nuclei appear to be well along in the process of ‘cup-from-cup’ separation before an increase in total DNA content from the single nucleus amount is clearly detected. The total amount of DNA increases from a low value approximating the average of single tumor nuclei in nuclei apparently beginning fission and reaches about two times the average nuclear content in nuclei that appear to have just completed fission.

### Example 2

### Syncytial Bell-Shaped Nuclei in Fetal Organogenesis

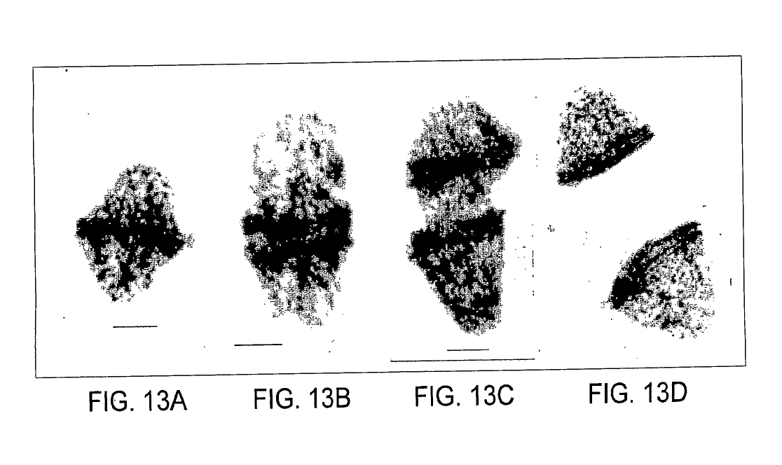

A series of previously unrecognized nuclear forms were identified in human fetal preparations that give rise to the bell-shaped nuclei. These forms were detected in the fifth week, as were the first tubular syncytia, which contain bell-shaped nuclei. Examples of these are shown in FIGS. 13A-D. This as an important finding marking the morphological transition from mitotic, spherical nuclei of early embryogenesis to the later amitotic, bell-shaped nuclei that represent the generative “stem” cell lineage of net growth and differentiation.

These findings are consistent across tissue types, as they have been observed in a series of tissue preparations including, for example, muscle, developing limbs, nervous tissue and visceral organs including the stomach, pancreas, bladder, lung and liver. The syncytia are found as clusters of ˜16-24 syncytia regularly spaced within the developing organ mass, each with ˜16 bell-shaped nuclei. Syncytia are apparent in the least developed human material available (˜5 weeks) and have disappeared by the thirteenth week. After the twelfth week the bell-shaped nuclei are regularly distributed in three dimensions in a manner peculiar to each organ.

FIG. 13A shows a nucleus with a condensation of ˜10% of the total DNA content as a “belt” around the long axis of spherical or slightly oval nuclei. FIG. 13B shows a nucleus in which two condensed nuclear “belts” appear to have separated but are still part of a single nucleus. FIG. 13C shows a pair of nuclei that appear to have arisen by fission of the two-belted nucleus of FIG. 13B. FIG. 13D shows that each syncytium contains a set of bells with a single pair of bells at its linear midpoint with mouths facing as in FIG. 13C. These images suggest to us that a series of symmetrical divisions create nuclei pushing away from a central pair. Syncytial structure is detected in groups as small as four bell-shaped nuclei.

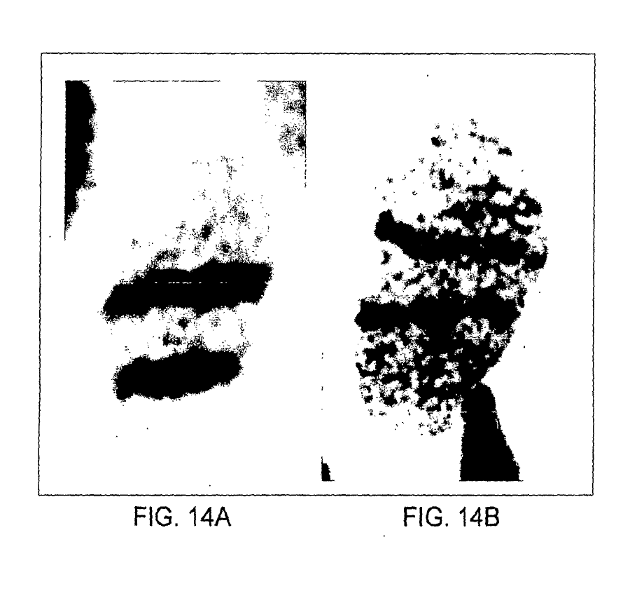

In studies of the nuclear morphotypes of carcinogenesis, nuclei showed similar belts—one or two around the long axis of oval nuclei—in small numbers in colonic adenomas (FIG. 14A) and adenocarcinomas (FIG. 14B). This finding confirms and extends support for the general hypothesis that oncogenesis shares many key phenotypic transitional steps of ontogenesis presenting, however, in reverse order of appearance.

### FISH Staining Specific for Human Centromeres.

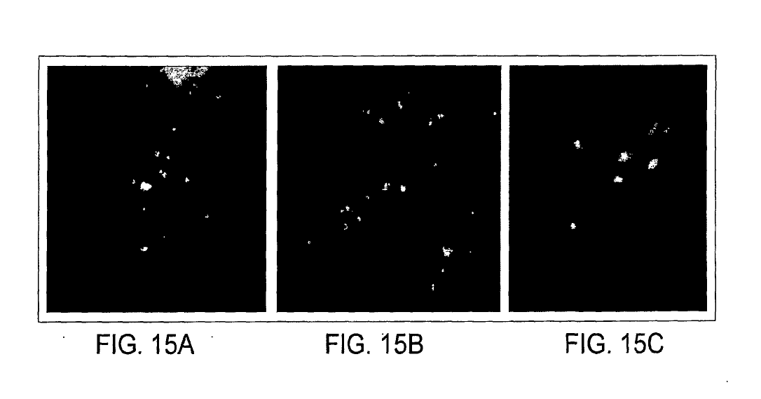

Extra-syncytial bell-shaped nuclei actually contain human DNA. Most centromeres are associated with the region of condensed DNA at the mouth of the bell-shaped nuclei in fetal samples. Interestingly, standard FISH protocols do not stain intra-syncytial bell-shaped or other shaped nuclei suggesting that the contractile element-containing sheath of the syncytium may block entry of the FISH reagents. FIG. 15 shows centromeres (in green) in spherical (FIG. 15A), “cigar”- (FIG. 15B) and bell- (FIG. 15C) shaped nuclei from tissues of human 12 weeks fetal colon.

It was also observed that the DNA content of sister nuclei are equal in fetal amitoses of bell-shaped nuclei, but they betray a marked degree of unequal DNA segregation in amitoses of bell-shaped nuclei in human tumors from multiple tissues of origin. Although no examples of amitotic fission among the bell-shaped nuclei of colonic preneoplastic polyps have been found, it is noted that the marked dispersion of DNA content among the dozens of bell-shaped nuclei found per polyp suggests that unequal DNA partitioning is a phenomena that is operative in preneoplasia as well as neoplasia, but not in fetal fissions of bell-shaped nuclei. These observations extend the observations of Virchow and Cohnheim that tumor tissue and embryonic tissue have similar histological features while also extending those of Boveri that tumor cells in mitosis betray a large fraction of aberrant chromosomes common to all cells of the tumor—suggesting an earlier common origin in unstable chromosomal formation or segregation.

### Nuclear Morphology in Mice.

All of the various forms of nuclei, in particular including the bell-shaped nuclei, pre-syncytial and syncytial forms in morphology almost identical to FIGS. 13A-D were found in tissue of fetal mice with the presyncytial forms first detected in 12.5 day, then in 14.5-16.5 days fetuses closely paralleling the period of organ definition in the fetal mouse. While these findings in the mouse are not surprising given the human observations, they open up a wide spectrum of possibilities of studies of organogenesis in non-human species not ethical or possible in humans.

In samples of quality fixed fetal discards ranging from the fifth through the sixteenth week of gestation, syncytia are no longer evident but bell-shaped nuclei are distributed in regular patterns throughout the growing organs.

### Example 3

Abundant syncytia and bell-shaped nuclei of the primitive gut are used to apply a series of histochemical procedures including FISH for defining the positions of chromosomes and chromosomal elements, various contractile molecules (e.g., actin) and other identifiable markers including those commonly denominated “stem cell markers”. Techniques described herein are applied to the task of collecting syncytia and individual nuclei using the ZEISS-P.A.L.M. microdissection instrument. The criterion of success is the collection of a series of samples homogeneous with regard to syncytial forms or nuclear morphotypes in numbers equal to or larger than 10,000 nuclear equivalents, numbers sufficient for scanning of cellular mRNAs, most common proteins and glycosaminoglycans.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for inhibiting tumor stem cell comprising targeting the tumor stem cell with an agent or treatment that chemically modifies a tumor stem cell-specific molecule, thereby preventing proliferation of tumor stem cells.

2. The method of claim 1, wherein the tumor stem cell-specific molecule is synthesized and segregated into daughter bell-shaped nuclei.

3. The method of claim 1, wherein the tumor stem cell-specific molecule is single-stranded DNA (ssDNA).

4. The method of claim 1, wherein the agent is a chemical agent.

5. The method of claim 1, wherein the agent is an enzyme.

6. The method of claim 1, wherein the treatment is radiation.

7. A method for inhibiting tumor growth in a patient comprising targeting a tumor stem cell in the patient with an agent or treatment that chemically modifies a tumor stem cell-specific molecule, thereby preventing proliferation of tumor stem cells.

8. The method of claim 7, wherein the tumor stem cell-specific molecule is synthesized and segregated into daughter bell-shaped nuclei.

9. The method of claim 7, wherein the tumor stem cell-specific molecule is single-stranded DNA (ssDNA).

10. The method of claim 7, wherein the agent is a chemical agent.

11. The method of claim 7, wherein the agent is an enzyme.

12. The method of claim 7, wherein the treatment is radiation.
